(19) United States
(12) Patent Application Publication
(10) Pub. No.: US 2013/0085177 A1
(43) Pub. Date: Apr. 4, 2013

(54) COSMETIC COMPOSITION BASED ON ELLAGIC ACID OR A DERIVATIVE THEREOF AND ON A PARTICULAR MIXTURE OF SURFACTANTS

(71) Applicants: Tiphaine Derkx, Paris (FR); Laurence Richet, Presles (FR)

(72) Inventors: Tiphaine Derkx, Paris (FR); Laurence Richet, Presles (FR)

(21) Appl. No.: 13/582,722

(22) PCT Filed: Mar. 1, 2011
(86) PCT No.: PCT/EP2011/053013

§ 371 Date: Dec. 17, 2012

Related U.S. Application Data
(60) Provisional application No. 61/313,851, filed on Mar. 15, 2010.
(60) Provisional application No. 61/313,853, filed on Mar. 15, 2010.

(30) Foreign Application Priority Data
Mar. 1, 2010 (FR) 1051442
Mar. 1, 2010 (FR) 1051445

(51) Int. Cl. A61K 8/49; A61Q 5/00

(52) U.S. Cl. CPC A61K 8/498; A61Q 5/006
USPC 514/453

(57) ABSTRACT

The invention relates to a cosmetic composition comprising, in a cosmetically acceptable medium: (i) from 0.2% to 10% by weight of one or more compounds selected from ellagic acid, ethers thereof, ellagic acid salts and ellagic acid ether salts, and mixtures thereof, and (ii) a system of surfactants selected from the following mixtures comprising: (iii) one or more anionic surfactants selected from ethoxylated alkyl sulphates, the corresponding acids, and mixtures thereof, and (iv) one or more anionic surfactants different from the abovementioned anionic surfactant(s) (iii), and (v) one or more anionic surfactants and (vi) one or more amphoteric or zwitterionic surfactants, in a weight ratio of the amount of anionic surfactant(s) to the amount of amphoteric or zwitterionic surfactant(s) of greater than 3. The invention also relates to a cosmetic treatment method using such a composition and to the use of said composition for washing keratin fibres.

The present invention relates to a cosmetic composition comprising, in particular proportions, at least one compound selected from ellagic acid, ethers thereof, the salts of these compounds, and mixtures thereof, and a system of particular surfactants.

The present invention also relates to a cosmetic treatment method using said composition and to the use of said composition for washing keratin fibres, in particular human keratin fibres such as the hair.

Ellagic acid is known in the prior art for its use as an active agent for preventing hair turning grey, or an antioxidant, depigmenting or else anti-pollution active agent. It can also be used as an anti-dandruff agent.

The problems of dandruff affect up to 50% of the world's population. They affect both men and women and are perceived as having a very negative psychosocial impact. The appearance of dandruff is bothersome both from an aesthetic point of view and because of the annoyances that it causes (itching, redness, etc.), with the result that many people confronted with this problem to varying degrees wish to be free of it in an effective and permanent manner.

Dandruff corresponds to excessive and visible desquamation of the scalp resulting from excessively rapid multiplication of the epidermal cells and their abnormal maturation. This phenomenon can be caused in particular by microtraumas of physical or chemical nature, such as hair treatments which are too aggressive, extreme climatic conditions, nervousness, diet, fatigue or pollution, but it has been demonstrated that dandruff conditions most commonly result from a disorder of the microflora of the scalp, and more particularly from excessive colonization by a fungus which belongs to the family of yeasts of the Malassezia genus (formerly known as Pytirosporum) and which is naturally present on the scalp.

It has been observed that since ellagic acid and derivatives thereof have very low solubility in water and in the solvents and oils commonly used in cosmetic compositions, these compounds have the drawback that they can be formulated virtually solely in dispersed form.

It is therefore difficult to homogeneously incorporate ellagic acid and derivatives thereof into cosmetic compositions, in particular into washing compositions which comprise an aqueous medium, without creating the formation of aggregates that can cause instability of the composition over time and a loss of effectiveness.

Thus, there is a need to have a washing base which promotes the dispersion of ellagic acid and of derivatives thereof by limiting the formation of aggregates, and which allows a very good distribution of these compounds during application and therefore optimum effectiveness, while at the same time retaining good detergence, good usage properties and good cosmetic properties.

Washing bases based on soaps of fatty acids or isolated surfactants do not make it possible to obtain completely satisfactory dispersions of ellagic acid or of derivatives thereof and/or result in insufficient effectiveness and/or usage properties and/or cosmetic properties.

The applicant has now found, surprisingly, that by combining, in particular proportions, at least one compound selected from ellagic acid, ethers thereof, ellagic acid salts and ellagic acid ether salts, with a particular mixture of anionic surfactants or of anionic and amphoteric or zwitterionic surfactants, it is possible to obtain a washing composition for keratin fibres which is stable over time, which exhibits a good dispersion of ellagic acid and/or of derivatives thereof, and good usage and detergence properties, which confers good distribution of the ellagic acid and derivatives thereof on the hair and the scalp, and which is particularly effective, in particular as an anti-dandruff composition.

In particular, it has been found that, when such a composition is used, it distributes easily on the hair and the scalp, and generates an abundant and soft foam that can be readily removed with water, resulting in soft, smooth, shiny clean hair. The composition according to the invention also exhibits a particularly notable anti-dandruff effectiveness, especially with repeated applications.

The composition according to the invention is stable over time and does not result in the formation of aggregates. In particular, it has a satisfactory storage stability both at ambient temperature (25° C.) and at higher temperature (37 or 45° C., for example).

A subject of the present invention is therefore a cosmetic composition comprising, in a cosmetically acceptable medium:
(i) from 0.2% to 10% by weight of one or more compounds selected from ellagic acid, ethers thereof, ellagic acid salts and ellagic acid ether salts, and mixtures thereof, and
(ii) a system of surfactants selected from the following mixtures comprising:
(iii) one or more anionic surfactants selected from ethoxylated alkyl sulphates, the corresponding acids, and mixtures thereof, and (iv) one or more anionic surfactants different from the abovementioned anionic surfactant(s) (iii), and
(v) one or more anionic surfactants and (vi) one or more amphoteric or zwitterionic surfactants, in a weight ratio of the amount of anionic surfactant(s) to the amount of amphoteric or zwitterionic surfactant(s) of greater than 3.

Another subject of the invention relates to a cosmetic treatment method for keratin fibres, in particular for washing keratin fibres, using said composition.

A subject of the invention is also the use of said composition for washing keratin fibres.

Other features, aspects, subjects and advantages of the present invention will emerge more clearly on reading the description and the examples which follow.

The composition according to the invention comprises one or more compounds (i) selected from ellagic acid, ethers thereof, ellagic acid salts, and ellagic acid ether salts.

Ellagic acid, also known as: 2,3,7,8-tetrahydroxy(1)benzo-pyrano(5,4,3-cde)(1)benzopyran-5,10-dione, is a well-known molecule present in the plant kingdom. Reference may be made to the publication of the Merck Index 20th edition (1996), No. 3588.

Ellagic acid has the following chemical formula:

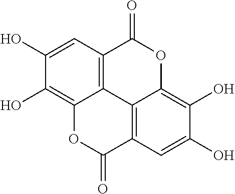 [SMILES: O=c1oc2c(O)c(O)cc3c(=O)oc4c(O)c(O)cc1c4c23]

which comprises four fused rings.

Ellagic acid is commercially available, especially from the company Sigma, France.

Document FR-A-1 478 523 discloses a process for purifying ellagic acid and also the purified ellagic acids obtained via such a process.

The ellagic acid ether(s) that can be used according to the invention is (are) preferably selected from the mono-, di-, tri- or polyethers obtained by etherification of one or more hydroxyl groups (one of the four OH groups of ellagic acid) of ellagic acid to give one or more OR groups, R being selected from C₂-C₂₀ alkyl groups, polyoxyalkylene groups, and in particular polyoxyethylene and/or polyoxypropylene groups, and groups derived from one or more monosaccharides or polysaccharides, such as, for example, the group having the following formula:

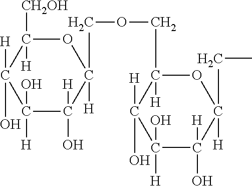

In the case of the di-, tri- or polyethers of ellagic acid, the R groups as defined above may be identical or different.

Such ethers are described in U.S. Pat. No. 5,073,545. Preferably, these ellagic acid ethers are selected from 3,4-di-O-methyl ellagic acid, 3,3′,4-tri-O-methyl ellagic acid and 3,3′-di-O-methyl ellagic acid.

The ellagic acid salt(s) and/or the ellagic acid ether salt(s) that can be used according to the invention are preferably selected from the salts of alkali or alkaline-earth metals, such as sodium, potassium, calcium and magnesium, the ammonium salt and the amine salts, such as the salts of triethanolamine, of monoethanolamine, of arginine and of lysine. Preferably, the ellagic acid salt(s) and/or the ellagic acid ether salt(s) that can be used according to the invention are selected from the alkali or alkaline-earth metal salts, in particular the sodium, potassium, calcium or magnesium salts.

Among all the compounds (i) mentioned, ellagic acid or a salt thereof is preferably used.

The composition according to the invention preferably comprises from 0.2% to 5% by weight, and even better still from 0.2% to 2% by weight of compound(s) (i), relative to the total weight of the composition.

The composition according to the invention comprises a system of particular surfactants (ii) as mentioned above.

The term “anionic surfactant” means a surfactant comprising, as ionic or ionizable groups, only anionic groups. These anionic groups are preferably selected from CO₂H, CO₂⁻, SO₃H, SO₃⁻, OSO₃H, OSO₃⁻, O₂PO₂H, O₂PO₂H⁻ and O₂PO₂²⁻ groups.

According to a first embodiment, the composition according to the invention comprises as system of surfactants, (iii) one or more anionic surfactants selected from ethoxylated alkyl sulphates, the corresponding acids, and mixtures thereof, and (iv) one or more anionic surfactants different from the abovementioned anionic surfactant(s) (iii). The ethoxylated alkyl sulphate(s) that can be used in the composition according to the invention can be preferably selected from ethoxylated (C₆-C₂₄)alkyl sulphates, in particular from ethoxylated (C₈-C₂₀)alkyl sulphates, and even better still ethoxylated (C₁₂-C₁₄)alkyl sulphates, such as ethoxylated lauryl sulphate.

The alkyl group of these compounds may be linear or branched. The alkyl group is preferably linear.

The number of ethylene oxide groups per molecule of ethoxylated alkyl sulphate will preferably range from 1 to 50, better still from 1 to 10, and even better still from 1 to 5. Even more preferably, this number ranges from 2 to 3.

The constituent salt(s) of the ethoxylated alkyl sulphate can be selected from alkali metal salts, such as the sodium salt, ammonium salts, amine salts, and in particular aminoalcohol salts, and alkaline-earth metal salts, such as the magnesium salt.

By way of example of aminoalcohol salts, mention may in particular be made of monoethanolamine, diethanolamine and triethanolamine salts, monoisopropanolamine, diisopropanolamine or triisopropanolamine salts, and 2-amino-2-methyl-1-propanol, 2-amino-2-methyl-1,3-propanediol and tris(hydroxymethyl)aminomethane salts.

Alkali metal or alkaline-earth metal salts, and in particular sodium or magnesium salts, are preferably used.

Among all the anionic surfactants (iii) mentioned, ethoxylated sodium lauryl sulphate comprising from 2 to 3 mol of ethylene oxide is preferably used.

In this embodiment, the composition according to the invention preferably comprises from 0.1% to 50% by weight, in particular from 2% to 20% by weight, even better still from 4% to 15%, and even better still from 5% to 15% by weight of anionic surfactant(s) (iii), relative to the total weight of the composition.

The anionic surfactant(s) (iv) different from the ethoxylated alkyl sulphates and from their corresponding acids, that can be used according to the invention, can be selected from the anionic surfactants normally used in the cosmetics industry, other than the ethoxylated alkyl sulphates and their corresponding acid forms (iii).

By way of example of anionic surfactants (iv) that can be used in the composition according to the invention, mention may be made of nonethoxylated alkyl sulphates, alkylamido ether sulphates, alkylaryl polyether sulphates, monoglyceride sulphates, alkyl sulphonates, alkylamide sulphonates, alkylarylsulphonates, alpha-olefin sulphonates, paraffin sulphonates, alkylsulphosuccinates, alkyl ether sulphosuccinates, alkylamide sulphosuccinates, alkyl sulphoacetates, acylsarcosinates, acylglutamates, alkyl sulphosuccinamates, acyl isethionates and N-acyltaurates; salts of alkyl monoesters of polyglycoside-polycarboxylic acids, acyllactylates, salts of D-galactosiduronic acids, salts of alkyl ether carboxylic acids, salts of alkyl aryl ether carboxylic acids, salts of alkylamido ether carboxylic acids; and the corresponding non-salified forms of all these compounds; the alkyl and acyl groups of all these compounds containing from 6 to 24 carbon atoms and the aryl group denoting a phenyl group.

Some of these compounds may be oxyethylenated and then preferably comprise from 1 to 50 ethylene oxide units, more particularly from 1 to 10.

The salts of C₆₋₂₄ alkyl monoesters of polyglycoside-polycarboxylic acids can be selected from C₆₋₂₄ alkyl polyglycoside-citrates, C₆₋₂₄ alkyl polyglycoside-tartrates and C₆₋₂₄ alkyl polyglycoside-sulphosuccinates.

When the anionic surfactant(s) (iv) is (are) in salt form, it (they) can be selected from alkali metal salts, such as the sodium or potassium salt, and preferably the sodium salt, ammonium salts (NH₄⁺), amine salts, and in particular aminoalcohol salts, and alkaline-earth metal salts such as the magnesium salt.

By way of example of aminoalcohol salts, mention may in particular be made of monoethanolamine, diethanolamine and triethanolamine salts, monoisopropanolamine, diisopropanolamine or triisopropanolamine salts, and 2-amino-2-methyl-1-propanol, 2-amino-2-methyl-1,3-propanediol and tris(hydroxymethyl)aminomethane salts.

The alkali metal or alkaline-earth metal salts and in particular the sodium or magnesium salts are preferably used.

Among all the anionic surfactants (iv) mentioned, the salts of (C₆-C₂₄ alkyl)ether carboxylic acids and nonethoxylated (C₆-C₂₄)alkyl sulphates are preferably used.

In particular, nonethoxylated (C₆-C₂₄)alkyl sulphates are preferably used, and in particular sodium lauryl sulphate, magnesium lauryl sulphate or ammonium lauryl sulphate. More preferably, sodium lauryl sulphate is used.

In this embodiment, the composition according to the invention preferably comprises from 0.1% to 30% by weight, in particular from 1% to 20% by weight, and better still from 2% to 15% by weight of anionic surfactant(s) (iv), relative to the total weight of the composition.

In one preferred embodiment, the anionic surfactant(s) (iii) is (are) selected from ethoxylated (C₆-C₂₄)alkyl sulphates comprising from 1 to 50, preferably from 1 to 10, in particular from 1 to 5, and even better still from 2 to 3 ethylene oxide units; and the anionic surfactant(s) (iv) is (are) selected from (C₆-C₂₄)alkyl sulphates.

In this preferred embodiment, the concentrations of anionic surfactants (iii) and (iv) can be as described above.

Advantageously, in the composition according to the invention, the weight ratio of the amount of anionic surfactant(s) (iii) to the amount of anionic surfactant(s) (iv) ranges from 0.25 to 20, preferably from 0.5 to 10, better still from 1 to 5 and even better still from 1 to 2.5.

In this embodiment, the composition according to the invention can also comprise one or more additional surfactants selected from amphoteric or zwitterionic surfactants.

By way of amphoteric or zwitterionic surfactants that can be used, mention may be made of those described in the embodiment below.

While they are present, the amount of the amphoteric or zwitterionic surfactant(s) is preferably included in the range of from 0.01% to 20% by weight, even better still from 0.5% to 10% by weight, relative to the total weight of the composition.

According to a second embodiment, the composition according to the invention comprises, by way of system of surfactants, (v) one or more anionic surfactants, and (vi) one or more amphoteric or zwitterionic surfactants, in a weight ratio of the amount of anionic surfactant(s) to the amount of amphoteric or zwitterionic surfactant(s) of greater than 3.

By way of examples of anionic surfactants (v) that can be used in the composition according to the invention mention may be made of alkyl sulphates, alkyl ether sulphates, alkylamido ether sulphates, alkylaryl polyether sulphates, monoglyceride sulphates, alkyl sulphonates, alkylamide sulphonates, alkylarylsulphonates, alpha-olefin sulphonates, paraffin sulphonates, alkylsulphosuccinates, alkyl ether sulphosuccinates, alkylamide sulphosuccinates, alkyl sulphoacetates, acylsarcosinates, acylglutamates, alkyl sulphosuccinamates, acyl isethionates and N-acyltaurates; salts of alkyl monoesters of polyglycoside-polycarboxylic acids, acyllactylates, salts of D-galactosiduronic acids, salts of alkyl ether carboxylic acids, salts of alkyl aryl ether carboxylic acids, salts of alkylamido ether carboxylic acids; and the corresponding non-salified forms of all these compounds; the alkyl and acyl groups of all these compounds containing from 6 to 24 carbon atoms and the aryl group denoting a phenyl group.

Some of these compounds may be oxyethylenated and then preferably comprise from 1 to 50 ethylene oxide units.

The salts of C₆₋₂₄ alkyl monoesters of polyglycoside-polycarboxylic acids can be selected from C₆₋₂₄ alkyl polyglycoside-citrates, C₆₋₂₄ alkyl polyglycoside-tartrates and C₆₋₂₄ alkyl polyglycoside-sulphosuccinates.

When the anionic surfactant(s) (v) is (are) in salt form, it (they) can be selected from alkali metal salts, such as the sodium or potassium salt, preferably the sodium salt, ammonium salts, amine salts, and in particular amino alcohol salts, or alkaline-earth metal salts such as the magnesium salt.

By way of example of aminoalcohol salts, mention may in particular be made of monoethanolamine, diethanolamine and triethanolamine salts, monoisopropanolamine, diisopropanolamine or triisopropanolamine salts, and 2-amino-2-methyl-1-propanol, 2-amino-2-methyl-1,3-propanediol and tris(hydroxymethyl)aminomethane salts.

The alkali metal or alkaline-earth metal salts and in particular the sodium or magnesium salts are preferably used.

Among all the anionic surfactants (v) mentioned, (C₆-C₂₄)alkyl sulphates, (C₆-C₂₄)alkyl ether sulphates comprising from 2 to 50 ethylene oxide units, in particular in the form of alkali-metal, ammonium, aminoalcohol and alkaline-earth metal salts, or a mixture of these compounds, is preferably used.

In particular, (C₁₂-C₂₀)alkyl sulphates, (C₁₂-C₂₀)alkyl ether sulphates comprising from 2 to 20 ethylene oxide units, in particular in the form of alkali metal, ammonium, aminoalcohol and alkaline-earth metal salts, or a mixture of these compounds, is preferably used. Even better still, sodium lauryl ether sulphate comprising 2.2 mol of ethylene oxide is preferably used.

In this embodiment, the composition according to the invention preferably comprises from 0.1% to 50% by weight, in particular from 4% to 30% by weight, and even better still from 8% to 20% by weight of anionic surfactant(s) (v), relative to the total weight of the composition.

The amphoteric or zwitterionic surfactant(s) (vi) that can be used in the present invention can in particular be derivatives of secondary or tertiary aliphatic amines, which are optionally quaternized, in which the aliphatic group is a linear or branched chain containing from 8 to 22 carbon atoms, said amine derivatives containing at least one anionic group such as, for example, a carboxylate, sulfonate, sulphate, phosphate or phosphonate group.

Mention may in particular be made of (C₈₋₂₀)alkylbetaines, sulphobetaines, (C₈₋₂₀ alkyl)amido(C₂₋₈ alkyl)betaines or (C₈₋₂₀ alkyl)amido(C₂₋₈ alkyl)sulphobetaines. Preferably, the betaine amphoteric or zwitterionic surfactant(s) (vi) are selected from (C₈₋₂₀)alkylbetaines and (C₈₋₂₀ alkyl)amido(C₂₋₈ alkyl)betaines.

Among the optionally quaternized secondary or teriary aliphatic amine derivatives that can be used, as defined above, mention may also be made of the products of respective structures (A2) and (A3) below:

Rₐ—CONHCH₂CH₂—N⁺(R_{b})(R_{c})(CH₂COO⁻) (A2)

in which:
Rₐ represents a C₁₀-C₃₀ alkyl or alkenyl group derived from an acid
Rₐ—COOH preferably present in hydrolysed coconut oil, a heptyl group,
a nonyl group or an undecyl group,
R_{b} represents a beta-hydroxyethyl group, and
R_{c} represents a carboxymethyl group;
and

R_{a′}—CONHCH₂CH₂—N(B)(B′) (A3)

in which:
B represents —CH₂CH₂OX′,
B′ represents —(CH₂)_{z}—Y′, with z=1 or 2,
X′ represents the —CH₂—COOH, CH₂—COOZ′, —CH₂CH₂—COOH or —CH₂CH₂—COOZ′ group, or a hydrogen atom,
Y′ represents —COOH, —COOZ′, or the —CH₂—CHOH—SO₃H or —CH₂—CHOH—SO₃Z′ group,
Z′ represents an ion derived from an alkali or alkaline-earth metal, such as sodium, potassium or magnesium; an ammonium ion; or an ion derived from an organic amine and in particular from an aminoalcohol, such as mono-, di- and triethanolamine, mono-, di- or triisopropanolamine, 2-amino-2-methyl-1-propanol, 2-amino-2-methyl-1,3-propanediol and tris(hydroxymethyl)aminomethane, R_{a′} represents a C₁₀-C₃₀ alkyl or alkenyl group of an acid R_{a′}COOH preferably present in hydrolysed linseed oil or coconut oil, an alkyl group, which is in particular C₁₇, and its iso form, or an unsaturated C₁₇ group.

The compounds corresponding to formula (A3) are preferred. These compounds are also classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M concentrate.

Among all the amphoteric or zwitterionic surfactants (vi) mentioned above, (C₈₋₂₀ alkyl)betaines, (C₈₋₂₀ alkyl)amido(C₂₋₈ alkyl)-betaines, and mixtures thereof, are preferably used. More preferably, the amphoteric or zwitterionic surfactant(s) is (are) selected from cocoylamidopropylbetaine and cocoylbetaine.

In this embodiment, the composition according to the invention preferably comprises from 0.01% to 20% by weight, in particular from 0.5% to 10% by weight, and even better still from 1% to 5% by weight of amphoteric or zwitterionic surfactant(s) (vi), relative to the total weight of the composition.

In one preferred embodiment, the anionic surfactant(s) (v) is (are) selected from (C₆-C₂₄)alkyl sulphates and (C₆-C₂₄)alkyl ether sulphates comprising from 2 to 50 ethylene oxide units; and the amphoteric or zwitterionic surfactant(s) (vi) is (are) selected from (C₈₋₂₀ alkyl)betaines, (C₈₋₂₀ alkyl)amido(C₂₋₈ alkyl)betaines and cocoamphodiacetates.

In one particularly preferred embodiment, the anionic surfactant(s) (v) is (are) selected from (C₁₆)alkyl sulphates and (C₁₆)alkyl ether sulphates in particular in the form of sodium salts comprising from 2 to 10 ethylene oxide units; and the amphoteric or zwitterionic surfactant(s) (vi) is (are) selected from cocoylamidopropylbetaine and cocoylbetaine.

In each of the preferred and particularly preferred embodiments described above, the concentration of anionic surfactant(s) (v) and of amphoteric or zwitterionic surfactant(s) (vi) can be as described above.

Advantageously, the composition according to the invention can comprise a weight ratio of the amount of anionic surfactant(s) (v) to the amount of amphoteric or zwitterionic surfactant(s) (vi) of less than or equal to 100, better still less than or equal to 50, and even better still less than or equal to 20 and even more preferably less than 10.

The composition according to the invention may also comprise one or more thickeners.

For the purpose of the present invention, the term “thickener” means an agent which, introduced at 1% by weight into an aqueous solution or aqueous-alcoholic solution containing 30% by weight of ethanol, at pH=7, makes it possible to achieve a viscosity of at least 100 cPs, preferably of at least 500 cPs, at 25° C. and at a shear rate of 1 s⁻¹. This viscosity may be measured using a cone/plate viscometer (Haake R600 rheometer or the like).

The thickener(s) can be selected from sodium chloride, fatty acid amides obtained from a C₁₀-C₃₀ carboxylic acid (coconut acid monoisopropanolamide, diethanolamide or monoethanolamide, oxyethylenated carboxylic acid monoethanolamide alkyl ether), non-ionic cellulosic thickeners (hydroxyethylcellulose, hydroxypropyl-cellulose, carboxymethylcellulose), guar gum and non-ionic derivatives thereof (hydroxypropylguar), gums of microbial origin (xanthan gum, scleroglucan gum), crosslinked or non-crosslinked homopolymers and copolymers based on acrylic acid, on methacrylic acid or on acrylamidopropanesulphonic acid, and the associative polymers as described below, and mixtures thereof.

The associative polymer(s) that can be used according to the invention are water-soluble polymers capable, in an aqueous medium, of reversibly associating with one another or with other molecules.

Their chemical structure comprises hydrophilic zones and hydrophobic zones characterized by at least one fatty chain preferably comprising from 10 to 30 carbon atoms.

The associative polymer(s) that can be used according to the invention may be of anionic, cationic, amphoteric or non-ionic type, such as the polymers sold under the names Pemulen TR1 or TR2 by the company Goodrich (INCI: Acrylates/C₁₀₋₃₀ Alkyl Acrylate Crosspolymer), Salcare SC90 by the company Ciba, Aculyn 22, 28, 33, 44 or 46 by the company Rohm & Haas, and Elfacos T210 and T212 by the company Akzo.

Among all the thickeners mentioned, the thickener(s) is (are) preferably selected from acrylic acid-based or methacrylic acid-based homopolymers and copolymers, which are preferably crosslinked, and/or fatty acid amides obtained from a C₁₀-C₃₀ carboxylic acid.

Preferably, the cosmetic composition comprises from 0.1% to 20% by weight, and even better still from 0.2% to 10% by weight of thickener(s), relative to the total weight of the composition.

The composition according to the invention may also comprise one or more conditioning agents.

According to the present invention, the term “conditioning agent” denotes any compound that can improve the cosmetic properties of the hair, in particular the softness, disentangling, feel and static electricity.

Preferably, the conditioning agent is selected from the group comprising cationic polymers, cationic surfactants, silicones, such as organosiloxanes, linear or branched C₈-C₃₀ hydrocarbons, linear or branched C₈-C₃₀ fatty alcohols, esters of C₈-C₃₀ fatty acid and of C₁-C₃₀ alcohol, and in particular esters of C₈-C₃₀ fatty acid and of C₈-C₃₀ fatty alcohol, esters of C₁-C₇ acid or diacid and of C₈-C₃₀ fatty alcohol, ceramides or ceramide analogues, and mixtures of these compounds.

The term “cationic polymer” means a polymer that is positively charged when it is contained in the composition according to the invention. This polymer may bear one or more positive permanent charges or may contain one or more functions that are cationizable in the composition according to the invention.

The cationic polymer(s) that may be used as conditioning agents according to the present invention are preferably selected from polymers comprising primary, secondary, tertiary and/or quaternary amine groups forming part of the polymer chain or directly attached thereto, and having a molecular weight of between 500 and about 5 000 000 and preferably between 1000 and 3 000 000.

When the conditioning agent is a cationic polymer, it is preferably selected from those that contain units comprising primary, secondary, tertiary and/or quaternary amine groups that may either form part of the main polymer chain, or may be borne by a side substituent directly attached thereto.

Among the cationic polymers that may be mentioned more particularly are polymers of the polyamine, polyamino amide and polyquaternary ammonium type. These are known products. They are described, for example, in French patents Nos. 2 505 348 and 2 542 997.

Among these polymers, mention may be made of:

(1) homopolymers or copolymers derived from acrylic or methacrylic esters or amides and comprising at least one of the units of the following formulae:

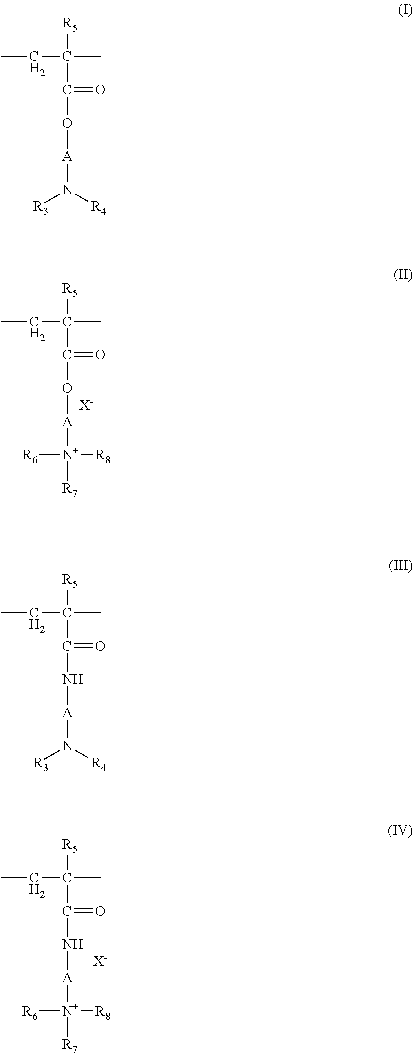

in which:

R₃ and R₄, which may be identical or different, represent a hydrogen atom or an alkyl group containing from 1 to 6 carbon atoms, and preferably methyl or ethyl;

R₅, which may be identical or different, denote a hydrogen atom or a CH₃ group;

A, which may be identical or different, represent a linear or branched alkyl group containing from 1 to 6 carbon atoms, preferably 2 or 3 carbon atoms, or a hydroxyalkyl group containing from 1 to 4 carbon atoms;

R₆, R₇ and R₈, which may be identical or different, represent an alkyl group containing from 1 to 18 carbon atoms or a benzyl group, and preferably an alkyl group containing from 1 to 6 carbon atoms;

X⁻ denotes an anion derived from a mineral or organic acid, such as a methosulphate anion or a halide such as chloride or bromide.

The copolymers of family (1) can also contain one or more units derived from comonomers which may be selected from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), mention may be made of:
copolymers of acrylamide and of dimethylaminoethyl methacrylate quaternized with dimethyl sulphate or with a dimethyl halide, such as the product sold under the name Hercofloc by the company Hercules,
the copolymers of acrylamide and of methacryloyloxyethyl-trimethylammonium chloride described, for example, in Patent Application EP-A-080 976 and sold under the name Bina Quat P 100 by the company Ciba Geigy,
the copolymer of acrylamide and of methacryloyloxyethyltrimethylammonium methosulphate sold under the name Reten by the company Hercules,
quaternized or non-quaternized vinylpyrrolidone/dialkyl-aminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinyl-pyrrolidone terpolymers, such as the product sold under the name Gaffix VC 713 by the company ISP,
vinylpyrrolidone/methacrylamidopropyldimethylamine copolymers sold in particular under the name Styleze CC 10 by ISP,
quaternized vinylpyrrolidone/dimethylaminopropylmethacryl-amide copolymers, such as the product sold under the name Gafquat HS 100 by the company ISP, and
crosslinked polymers of methacryloyloxy(C1-C4)alkyl(C1-C4)trialkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with a compound comprising an olefinic unsaturation, in particular methylenebisacrylamide. Use may more particularly be made of an acrylamide/methacryloyloxyethyltrimethylammonium chloride crosslinked copolymer (20/80 by weight) in the form of a dispersion containing 50% by weight of said copolymer in mineral oil. This dispersion is sold under the name Salcare® SC 92 by the company Ciba. A crosslinked methacryloyloxyethyltrimethylammonium chloride homopolymer, for example as a dispersion in mineral oil or in a liquid ester, can also be used. These dispersions are sold under the names Salcare® SC 95 and Salcare® SC 96 by the company Ciba;

(2) polymers constituted of piperazinyl units and of divalent alkylene or hydroxyalkylene groups containing straight or branched chains, optionally interrupted with oxygen, sulphur or nitrogen atoms or with aromatic or heterocyclic rings, and also the oxidation and/or quaternization products of these polymers. Such polymers are described, in particular, in French patents 2 162 025 and 2 280 361;

(3) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a bifunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508;

(4) the polyamino amide derivatives resulting from the condensation of polyalkylene polyamines with polycarboxylic acids, followed by alkylation with bifunctional agents. Mention may be made, for example, of adipic acid/dialkylaminohydroxyalkyl-dialkylenetriamine polymers in which the alkyl groups contain from 1 to 4 carbon atoms and preferably denote a methyl, ethyl or propyl group, and the alkylene groups contain from 1 to 4 carbon atoms and preferably denote the ethylene group. Such polymers are in particular described in French patent 1 583 363.

Among these derivatives, mention may be made more particularly of the adipic acid/dimethylaminohydroxypropyldiethylene-triamine polymers sold under the name Cartaretine F, F4 or F8 by the company Sandoz.

(5) The polymers obtained by reaction of a polyalkylene polyamine containing two primary amine groups and at least one secondary amine group with a dicarboxylic acid selected from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom being reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyldiethylenetriamine copolymer.

(6) cyclopolymers of alkyldiallylamine or of dialkyldiallylammonium, such as homopolymers or copolymers containing, as main constituent of the chain, units corresponding to formula (V) or (VI):

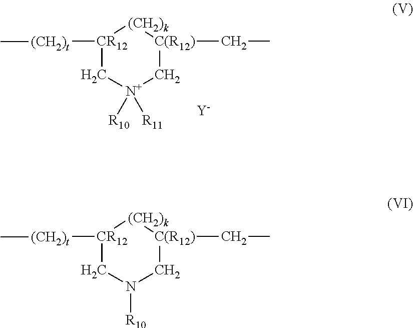

in which formulae k and t are equal to 0 or 1, the sum k+t being equal to 1; R₁₂ denotes a hydrogen atom or a methyl group; R₁₀ and R₁₁, independently of each other, denote an alkyl group having from 1 to 6 carbon atoms, a hydroxyalkyl group in which the alkyl group preferably has 1 to 5 carbon atoms, a lower (C₁-C₄) amidoalkyl group, or R₁₀ and R₁₁ can denote, together with the nitrogen atom to which they are attached, heterocyclic groups such as piperidinyl or morpholinyl; Y⁻ is an anion such as bromide, chloride, acetate, borate, citrate, tartrate, bisulphate, bisulphite, sulphate or phosphate. These polymers are described in particular in French patent 2 080 759 and in its Certificate of Addition 2 190 406.

R₁₀ and R₁₁, independently of each other, preferably denote an alkyl group containing from 1 to 4 carbon atoms.

Among the polymers defined above, mention may be made more particularly of the dimethyldiallylammonium chloride homopolymer sold under the name Merquat 100 by the company Nalco (and its homologues of low weight-average molecular mass) and the copolymers of diallyldimethylammonium chloride and of acrylamide, sold under the name Merquat 550.

(7) the quaternary diammonium polymer containing repeating units corresponding to formula (VII):

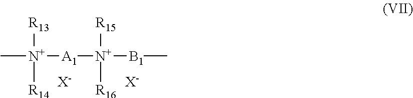

in which formula (VII):

R₁₃, R₁₄, R₁₅ et R₁₆, which may be identical or different, represent aliphatic, alicyclic or arylaliphatic groups containing from 1 to 20 carbon atoms or lower (C₁-C₄) hydroxyalkyl aliphatic groups, or alternatively R₁₃, R₁₄, R₁₅ and R₁₆, together or separately, constitute, with the nitrogen atoms to which they are attached, heterocycles optionally containing a second heteroatom other than nitrogen, or alternatively R₁₃, R₁₄, R₁₅ and R₁₆ represent a linear or branched C₁-C₆ alkyl group substituted with a nitrile, ester, acyl or amide group or a group —CO—O—R₁₇-D or —CO—NH—R₁₇-D or R₁₇ is an alkylene having from 1 to 10 carbon atoms and D a quaternary ammonium group;

A₁ and B₁ represent polymethylene groups containing from 2 to 20 carbon atoms which may be linear or branched, saturated or unsaturated, and which may contain, linked to or intercalated in the main chain, one or more aromatic rings or one or more oxygen, or sulphur atoms or sulphoxide, sulphone, disulphide, amino, alkylamino, hydroxyl, quaternary ammonium, ureido, amide or ester groups, and

X⁻ denotes an anion derived from a mineral or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene group, B₁ can also denote a group

—(CH₂)ₙ—CO-D-OC—(CH₂)ₚ—

in which:

n and p are integers ranging from 2 to 20 approximately,

D denotes:

a) a glycol residue of formula: —O—Z—O—, where Z denotes a linear or branched hydrocarbon-based group or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based group, or alternatively the divalent group —CH₂—CH₂—S—S—CH₂—CH₂—;

d) a ureylene group of formula: —NH—CO—NH—.

Preferably, X⁻ is an anion such as chloride or bromide.

These polymers generally have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may more particularly be made of polymers which are constituted of repeating units corresponding to formula (VIII):

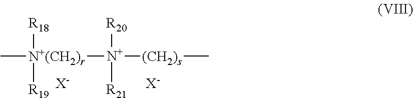

in which: R₁₈, R₁₉, R₂₀ and R₂₁, which may be identical or different, denote an alkyl or hydroxyalkyl group containing from 1 to 4 carbon atoms approximately, r and s are integers ranging from 2 to 20 approximately, and X⁻ is an anion derived from a mineral organic acid.

A compound of formula (VIII) which is particularly preferred is the compound for which R₁₈, R₁₉, R₂₀ and R₂₁ representent a methyl group and r=3, s=6 and X═Cl, referred to as hexadimethrine chloride according to the INCI nomenclature (CTFA).

(8) Polyquaternary ammonium polymers constituted of units of formula (IX):

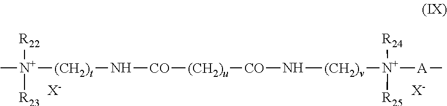

in which formula:

R₂₂, R₂₃, R₂₄ and R₂₅, which may be identical or different, represent a hydrogen atom or a methyl, ethyl, propyl, β-hydroxyethyl, β-hydroxypropyl or —CH₂CH₂(OCH₂CH₂)ₚOH group, where p is equal to 0 or to an integer ranging from 1 to 6, with the proviso that R₂₂, R₂₃, R₂₄ and R₂₅ do not simultaneously represent a hydrogen atom,

t and u, which may be identical or different, are integers between 1 and 6,

v is equal to 0 or to an integer between 1 and 34,

X⁻ denotes an anion such as a halide;

A denotes a group of a dihalide or preferably represents —CH₂—CH₂—O—CH₂—CH₂—.

Compounds of this kind are described in particular in Patent Application EP-A-122 324.

Among these products, mention may, for example, be made of the products Mirapol® A 15, Mirapol® AD1, Mirapol® AZ1 and Mirapol® 175 sold by the company Miranol.

(9) quaternary polymers of vinylpyrrolidone and of vinylimidazole, such as, for example, the products sold under the names Luviquat® FC 905, FC 550 and FC 370 by the company B.A.S.F.

(10) cationic polysaccharides, in particular cationic celluloses and cationic cellulose derivatives and cationic galactomannan gums.

Among the cationic polysaccharides, mention may more particularly be made of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are described in French patent 1 492 597. These polymers are also defined in the CTFA dictionary as quaternary ammoniums of hydroxyethylcellulose having reacted with an epoxide substituted with a trimethylammonium group.

The cationic cellulose copolymers or the cellulose derivatives grafted with a water-soluble quaternary ammonium monomer are described especially in U.S. Pat. No. 4,131,576, such as hydroxyalkylcelluloses, for instance hydroxymethyl-, hydroxyethyl- or hydroxypropylcelluloses grafted especially with a methacryloylethyltrimethylammonium, methacrylamidopropyl-trimethylammonium or dimethyldiallylammonium salt.

The cationic galactomannan gums are described more particularly in U.S. Pat. Nos. 3,589,578 and 4,031,307, in particular guar gums containing trialkylammonium cationic groups. Use is made, for example, of guar gums modified with a salt (e.g. chloride) of 2,3-epoxypropyltrimethylammonium.

Other cationic polymers that can be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The cationic proteins or protein hydrolysates are, in particular, chemically modified polypeptides bearing quaternary ammonium groups at the end of the chain, or grafted thereon. Their molecular mass may vary, for example, from 1500 to 10 000 and in particular from 2000 to 5000 approximately. Among these compounds, mention may be made especially of:
hydrolysates of collagen bearing triethylammonium groups, such as the products sold under the name Quat-Pro E by the company Maybrook and called, in the CTFA dictionary, Triethonium Hydrolyzed Collagen Ethosulphate;
hydrolysates of collagen bearing trimethylammonium and trimethylstearylammonium chloride groups, sold under the name Quat-Pro S by the company Maybrook and called, in the CTFA dictionary, Steartrimonium Hydrolyzed Collagen;
hydrolysates of animal proteins bearing trimethylbenzylammonium groups, such as the products sold under the name Crotein BTA by the company Croda and called, in the CTFA dictionary, Benzyltrimonium hydrolyzed animal protein;
hydrolysates of proteins bearing, on the polypeptide chain, quaternary ammonium groups comprising at least one alkyl group containing from 1 to 18 carbon atoms.

Among these protein hydrolysates, mention may be made, inter alia, of:
Croquat L, the quaternary ammonium groups of which comprise a C₁₂ alkyl group;
Croquat M, the quaternary ammonium groups of which comprise C₁₀-C₁₈ alkyl groups;
Croquat S, the quaternary ammonium groups of which comprise a C₁₈ alkyl group;
Crotein Q, the quaternary ammonium groups of which comprise at least one alkyl group containing from 1 to 18 carbon atoms.

These various products are sold by the company Croda.

Other quaternized proteins or hydrolysates are, for example, those corresponding to formula (X):

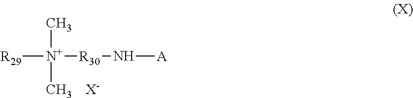

in which X⁻ is an anion of an organic or mineral acid, A denotes a protein residue derived from collagen protein hydrolysates, R₂₉ denotes a lipophilic group containing up to 30 carbon atoms, R₃₀ represents an alkylene group containing from 1 to 6 carbon atoms. Mention may, for example, be made of the products sold by the company Inolex, under the name Lexein QX 3000, called, in the CTFA dictionary, Cocotrimonium Collagen Hydrolysate.

Mention may also be made of quaternized plant proteins, such as wheat, corn or soya proteins: as quaternized wheat proteins, mention may be made of those sold by the company Croda under the names Hydrotriticum WQ or QM, called, in the CTFA dictionary, Cocodimonium hydrolysed wheat protein, Hydrotriticum QL, called, in the CTFA dictionary, Laurdimonium hydrolysed wheat protein, or else Hydrotriticum QS, called, in the CTFA dictionary, Steardimonium hydrolysed wheat protein.

Among all the cationic polymers that may be used in the context of the present invention, it is preferred to use the cationic cyclopolymers as defined above, in particular the dimethyldiallylammonium chloride homopolymers or copolymers sold under the names Merquat 100, Merquat 550 and Merquat S by the company Nalco, quaternary vinylpyrrolidone and vinylimidazole polymers, cationic polysaccharides and mixtures thereof.

The conditioning agent(s) that can be used according to the invention can be selected from cationic surfactants.

The term “cationic surfactant” means a surfactant which is positively charged when it is contained in the composition according to the invention. This surfactant may bear one or more positive permanent charges or may contain one or more functions that are cationizable in the composition according to the invention.

The cationic surfactant(s) that may be used as conditioning agents according to the present invention are preferably selected from primary, secondary or tertiary fatty amines, optionally polyoxyalkylenated, or salts thereof, and quaternary ammonium salts, and mixtures thereof.

The fatty amines generally comprise at least one C₈-C₃₀ hydrocarbon-based chain. Among the fatty amines that may be used according to the invention, examples that may be mentioned include stearylamidopropyldimethylamine and distearylamine.

Examples of quaternary ammonium salts that may especially be mentioned include:
those corresponding to general formula (XI) below:

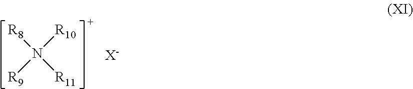

in which the groups R₈ to R₁₁, which may be identical or different, represent a linear or branched aliphatic group containing from 1 to 30 carbon atoms, or an aromatic group such as an aryl or an alkylaryl group, at least one of the groups R₈ to R₁₁ denoting a group containing from 8 to 30 carbon atoms, preferably from 12 to 24 carbon atoms. The aliphatic groups can comprise heteroatoms such as, in particular, oxygen, nitrogen, sulphur and halogens. The aliphatic groups are, for example, selected from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion selected from the group of halides, phosphates, acetates, lactates, (C₁-C₄)alkyl sulphates and (C₁-C₄)alkyl or (C₁-C₄)alkylaryl sulphonates.

Among the quaternary ammonium salts of formula (XI), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, the palmitylamidopropyltrimethylammonium salt, the stearamidopropyltrimethylammonium salt, the stearamidopropyldimethylcetearylammonium salt, or the stearamidopropyldimethyl(myristyl acetate)ammonium salt sold under the name Ceraphyl® 70 by the company Van Dyk. It is particularly preferred to use the chloride salts of these compounds;
quaternary ammonium salts of imidazoline, such as, for example, those of formula (XII) below:

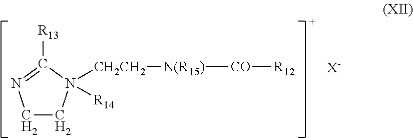

in which R₁₂ represents an alkenyl or alkyl group containing from 8 to 30 carbon atoms, for example tallow fatty acid derivatives, R₁₃ represents a hydrogen atom, a C₁-C₄ alkyl group or an alkenyl or alkyl group containing from 8 to 30 carbon atoms, R₁₄ represents a C₁-C₄ alkyl group, R₁₅ represents a hydrogen atom or a C₁-C₄ alkyl group, X⁻ is an anion selected from the group of halides, phosphates, acetates, lactates, alkyl sulphates, alkyl sulphonates or alkylaryl sulphonates, the alkyl and aryl groups of which preferably comprise, respectively, from 1 to 20 carbon atoms and from 6 to 30 carbon atoms. Preferably, R₁₂ and R₁₃ denote a mixture of alkenyl or alkyl groups containing from 12 to 21 carbon atoms, for example tallow fatty acid derivatives, R₁₄ denotes a methyl group and R₁₅ denotes a hydrogen atom. Such a product is, for example, sold under the name Rewoquat® W 75 by the company Rewo;
diquaternary or triquaternary ammonium salts of formula (XIII):

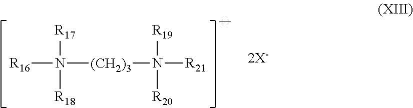

in which R₁₆ denotes an alkyl group containing from approximately 16 to 30 carbon atoms, which is optionally hydroxylated and/or interrupted with one or more oxygen atoms, R₁₇ is selected from hydrogen or an alkyl group containing from 1 to 4 carbon atoms or the following group:

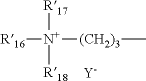

R′₁₆, R′₁₇, R′₁₈, R₁₈, R₁₉, R₂₀ and R₂₁, which may be identical or different, are selected from hydrogen or an alkyl group containing from 1 to 4 carbon atoms, and X⁻ and Y⁻ are anions in particular selected from the group of halides, acetates, phosphates, nitrates and (C₁-C₆)alkyl sulphates, in particular methyl sulphate or ethyl sulphate. Such compounds are, for example, Finquat CT-P offered by the company Finetex (Quaternium-89), Finquat CT offered by the company Finetex (Quaternium 75) and Condicate CT offered by the company Innospec Active Chemicals (Quaternium-75);
quaternary ammonium salts containing at least one ester function, such as those of formula (XIV) below:

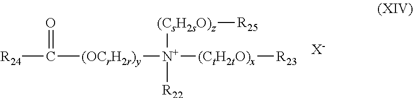

in which:

R₂₂ is selected from C₁-C₆ alkyl groups and C₁-C₆ hydroxyalkyl or dihydroxyalkyl groups;
R₂₃ is selected from:
the group

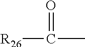

the groups R₂₇ which are linear or branched, saturated or unsaturated C₁-C₂₂ hydrocarbon-based groups,
a hydrogen atom,
R₂₅ is selected from:
the group

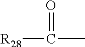

the groups R₂₉ which are linear or branched, saturated or unsaturated C₁-C₆ hydrocarbon-based groups,
a hydrogen atom,

R₂₄, R₂₆ and R₂₈, which may be identical or different, are selected from linear or branched, saturated or unsaturated C₇-C₂₁ hydrocarbon-based groups;

r, s and t, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₂₃ denotes R₂₇ and that when z is 0, then R₂₅ denotes R₂₉.

The R₂₂ alkyl groups may be linear or branched and more particularly linear.

Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may be long and contain from 12 to 22 carbon atoms, or short and contain from 1 to 3 carbon atoms.

When R₂₅ is a hydrocarbon-based group R₂₉, it preferably contains from 1 to 3 carbon atoms.

Advantageously, R₂₄, R₂₆ and R₂₈, which may be identical or different, are selected from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which may be identical or different, are 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide) or an alkyl sulphate, more particularly methyl sulphate. However, methanesulphonate, phosphate, nitrate, tosylate, an anion derived from an organic acid, such as acetate or lactate, or any other anion compatible with the ammonium containing an ester function, may be used.

The anion X⁻ is even more particularly chloride or methyl sulphate.

The ammonium salts more particularly used in the composition according to the invention are the ammonium salts of formula (XIV) in which:

R₂₂ denotes a methyl or ethyl group,

x and y are equal to 1;

z is equal to 0 or 1;

r, s and t are equal to 2;
R₂₃ is selected from:
the group

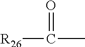

methyl, ethyl or C₁₄-C₂₂ hydrocarbon-based groups,
a hydrogen atom;
R₂₅ is selected from:
the group

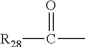

a hydrogen atom;

R₂₄, R₂₆ and R₂₈, which may be identical or different, are selected from linear or branched, saturated or unsaturated C₁₃-C₁₇ hydrocarbon-based groups, and are preferably from linear or branched, saturated or unsaturated C₁₃-C₁₇ alkyl or alkenyl groups.

The hydrocarbon-based groups are advantageously linear.

Mention may be made, for example, of the compounds of formula (XIV) such as the diacyloxyethyldimethylammonium, diacyloxyethylhydroxyethylmethylammonium, monoacyloxyethyl-dihydroxyethylmethylammonium, triacyloxyethylmethylammonium and monoacyloxyethylhydroxyethyldimethylammonium salts (chloride or methyl sulphate in particular), and mixtures thereof. The acyl groups preferably contain 14 to 18 carbon atoms and are obtained more particularly from a plant oil such as palm oil or sunflower oil. When the compound contains several acyl groups, these groups may be identical or different.

These products are obtained, for example, by direct esterification of triethanolamine, triisopropanolamine, an alkyldi-ethanolamine or an alkyldiisopropanolamine, which are optionally oxyalkylenated, with C₁₀-C₃₀ fatty acids or with mixtures of C₁₀-C₃₀ fatty acids of plant or animal origin, or by transesterification of the methyl esters thereof. This esterification is followed by a quaternization using an alkylating agent such as an alkyl halide (preferably a methyl or ethyl halide), a dialkyl sulphate (preferably dimethyl or diethyl sulphate), methyl methanesulphonate, methyl para-toluenesulphonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention may contain, for example, a mixture of quaternary ammonium salts of mono-, di- and triesters with a weight majority of diester salts.

Mixtures of ammonium salts that can be used include, for example, the mixture containing 15% to 30% by weight of acyloxyethyldihydroxyethylmethylammonium methyl sulphate, 45% to 60% of diacyloxyethylhydroxyethylmethylammonium methyl sulphate and 15% to 30% of triacyloxyethylmethylammonium methyl sulphate, the acyl groups containing from 14 to 18 carbon atoms and being obtained from palm oil, which is optionally partially hydrogenated.

It is also possible to use the ammonium salts containing at least one ester function that are described in U.S. Pat. No. 4,874,554 and U.S. Pat. No. 4,137,180.

The particularly preferred cationic surfactant(s) that can be used according to the invention is (are) selected from the compounds of formula (XI) or of formula (XIV), methyl (C₉-C₁₉)alkyl(C₁₀-C₂₀)alkylaminoethylimidazolium salts and stearamidopropyldimethyl-amine.

Among all the cationic surfactants that may be present in the composition according to the invention, cetyltrimethylammonium, behenyltrimethylammonium, di(palmitoyloxyethyl)hydroxyethyl-methylammonium, di(stearoyloxyethyl)hydroxyethylmethylammonium and methyl(C₉-C₁₉)alkyl(C₁₀-C₂₀)alkylamidoethylimidazolium salts, the stearamidopropyltrimethylammonium salt, the stearamidopropyl-dimethylamine salt and the stearamidopropyldimethylcetearyl-ammonium salt, and mixtures thereof, are preferably selected.

Among the silicones that can be used as conditioning agents in accordance with the present invention, mention may be made, in a nonlimiting manner, of:

I. Volatile Silicones:

These silicones have a boiling point of between 60° C. and 260° C. Among the silicones of this type that are mentioned are:

(a) cyclic silicones containing from 3 to 7 silicon atoms, and preferably 4 to 5.

These are, for example, octamethylcyclotetrasiloxane sold under the name Volatile Silicone 7207® by the company Union Carbide or Silbione 70045 V2® by the company Rhône-Poulenc, decamethylcyclopentasiloxane sold under the name Volatile Silicone 7158® by the company Union Carbide, Silbione 70045 V5® by the company Rhône-Poulenc, and mixtures thereof. Mention is also made of cyclopolymers of the dimethylsiloxane/methylalkylsiloxane type, such as Volatile Silicone FZ 3109® sold by the company Union Carbide, which is a dimethylsiloxane/methyloctylsiloxane cyclopolymer;

(b) linear volatile silicones containing 2 to 9 silicon atoms and having a viscosity of less than or equal to 5×10⁻⁶ m²/s at 25° C.

An example is the hexamethyldisiloxane sold under the name Silbione 70041 V0,65® by the company Rhône-Poulenc. This type of product is described in the article by Todd & Byers “Volatile silicone fluids for cosmetics”, Cosmetics and Toiletries, Vol. 91, January 76, pages 27 32.

II. Non-Volatile Silicones

These silicones are mainly constituted by polyalkylsiloxanes, polyarylsiloxanes polyalkylarylsiloxanes and organomodified polysiloxanes, and mixtures thereof. They may be in the form of oils, gums and resins.

Among the polyalkylsiloxanes, mention may be made mainly of linear polydimethylsiloxanes with a viscosity of greater than 5×10⁻⁶ m²/s, and preferably less than 2.6 m²/s, i.e.:
containing trimethylsilyl end groups, for instance, and in a nonlimiting manner, the Silbione® oils of the 70047 series sold by the company Rhône-Poulenc, the Wacker Belsil DM 60000 oil from Wacker or certain Viscasil® from the company General Electric,
containing trihydroxysilyl end groups, such as the oils of the 48 V® series from the company Rhône-Poulenc.

In this class of polyalkylsiloxanes, mention may also be made of the polyalkylsiloxanes sold by the company Goldschmidt under the names Abil Wax 9800® and Abil Wax 9801®, which are poly(C₁₋₂₀)alkylsiloxanes.

Among the polyalkylarylsiloxanes, mention may be made of linear and/or branched polydimethylphenylsiloxanes and polydimethyldiphenylsiloxanes, with a viscosity from 10⁻⁵ to 5×10⁻² m²/s, for instance:
the oil Rhodorsil® 763 from Rhône-Poulenc,
the Silbione® oils of the 70641 series from Rhône-Poulenc, such as the oils Silbione 70641 V30® and Silbione 70641 V200®,
the product DC 556® Cosmetic Grade Fluid from Dow Corning,
the silicones of the PK series from Bayer, such as PK200,
the silicones of the PN and PH series from Bayer, such as the products PN1000® and PH1000®;
certain oils of the SF series from General Electric, such as SF 1250®, SF 1265®, SF 1154® and SF 1023®. The silicone gums in accordance with the present invention are polydiorganosiloxanes with a high number-average molecular mass of between 200 000 and 1 000 000, used alone or as a mixture in a solvent selected from volatile silicones, polydimethylsiloxane (PDMS) oils, polyphenylmethylsiloxane (PPMS) oils, isoparaffins, methylene chloride, pentane, dodecane, tridecane and tetradecane, or mixtures thereof.

Mention is made, for example, of the compounds having the following structures:
poly[(dimethylsiloxane)/(methylvinylsiloxane)] gums,
poly[(dimethylsiloxane)/(diphenylsiloxane)] gums,
poly[(dihydrogenodimethylsiloxane)/(divinylsiloxane)] gums,
poly[(dimethylsiloxane)/(phenylmethylsiloxane)] gums,
poly[(dimethylsiloxane)/(diphenylsiloxane)/(methylvinyl-siloxane)] gums.

The Mirasil DM 300 000 gum from the company Rhodia may be mentioned.

Mention may also be made, for example, in a nonlimiting manner, of the following mixtures:

1) mixtures formed from a polydimethylsiloxane hydroxylated at the end of the chain (dimethiconol according to the CTFA nomenclature), and from a cyclic polydimethylsiloxane (cyclomethicone according to the CTFA nomenclature), such as the products Q2 1401® or Dow Corning 1501 Fluid sold by the company Dow Corning;

2) mixtures formed from a polydimethylsiloxane gum with a cyclic silicone, such as the product SF 1214 Silicone Fluid® from General Electric, which is an SE 30® gum of MW 500 000 (−Mₙ) dissolved in SF 1202 Silicone Fluid® (decamethylcyclopentasiloxane);

3) mixtures of two PDMSs of different viscosities, especially of a PDMS gum and a PDMS oil, such as the products SF 1236® and CF 1241® from the company General Electric. The product SF 1236® is a mixture of an SE 30® gum defined above, with a viscosity of 20 m²/s, and of an SF 96® oil with a viscosity of 5×10⁶ m²/s (15% SE 30® gum and 85% SF 96® oil).

The product CF 1241® is a mixture of an SE 30® gum (33%) and of a PDMS (67%), with a viscosity of 10⁻³ m²/s.

The organopolysiloxane resins that may be used in accordance with the invention are crosslinked siloxane systems containing the following units: R₂SiO_{2/2}, RSiO_{3/2} and SiO_{4/2} in which R represents a hydrocarbon-based group containing 1 to 6 carbon atoms or a phenyl group. Among these products, those which are particularly preferred are those in which R denotes a (C₁-C₄) lower alkyl group or a phenyl group.

Among these resins, mention may be made of the product sold under the name Dow Corning 593® or those sold under the names Silicone Fluid SS 4230 and Silicone Fluid SS 4267 by the company General Electric and which are dimethyl/trimethylpolysiloxanes.

The organomodified silicones in accordance with the present invention are silicones as defined above, comprising, in their general structure, one or more organofunctional groups directly attached to the siloxane chain or attached by means of a hydrocarbon-based group.

Mention is made, for example, of the silicones comprising:

a) perfluoro groups such as trifluoroalkyls, for instance those sold by the company General Electric under the names FF.150 Fluorosilicone Fluid® or by the company Shin-Etsu under the names X-22-819®, X-22-82®, X-22-821® and X-22-822®;

b) hydroxyacylamino groups, for instance those described in patent application EP 0 342 834 and in particular the silicone sold by the company Dow Corning under the name Q2-8413®;

c) thiol groups, as in the silicones X 2-8360® from the company Dow Corning or GP 72A® and GP 71® from Genesee;

d) non-quaternized amino groups, such as GP 4 Silicone Fluid® from Genesee, GP 7100® from Genesee, Q2 8220® from Dow Corning, AFL 40® from union Carbide or the silicone known as Amodimethicone in the CTFA dictionary;

e) carboxylate groups, for instance the products described in patent EP 186 507 from Chisso Corporation;

f) hydroxylated groups, such as polyorganosiloxanes comprising a hydroxyalkyl function, described in Patent Application FR 85 16334, corresponding to formula (XV) below:

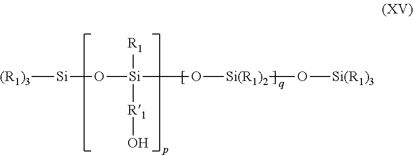

in which:
the R₁ groups, which may be identical or different, are selected from methyl and phenyl groups, at least 60 mol % of the R₁ groups being methyl;
the R′₁ group is a divalent hydrocarbon-based C₂-C₁₈ alkylene link;
p is between 1 and 30 inclusive;
q is between 1 and 150 inclusive.

Mention may most particularly be made of the product sold by Dow Corning under the name DC 190;

g) alkoxylated groups, for instance in the silicone copolymer F 755® from SWS Silicones and the products Abil Wax 2428®, Abil Wax 2434® and Abil Wax 2440® from the company Goldschmidt;

h) acyloxyalkyl groups, for instance the polyorganopolysiloxanes described in Patent Application FR 88 17433, corresponding to formula (XVI) below:

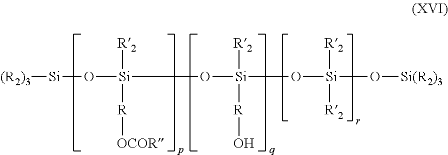

in which:
R₂ denotes methyl, phenyl, OCOR″ or hydroxyl, it being possible for only one of the R₂ per silicon atom to be OH;
R′₂ denotes methyl or phenyl, at least 60 mol % of the R₂ and
R′₂ groups together being methyl;
R″ denotes C₈-C₂₀ alkyl or alkenyl;
R denotes a divalent, linear or branched C₂-C₁₈ hydrocarbon-based alkylene;
r is between 1 and 120 inclusive;
p is between 1 and 30 inclusive;
q is equal to 0 or is less than 0.5 p, p+q being between 1 and 30 inclusive;

the polyorganosiloxanes of formula (XVI) can comprise:

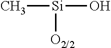

groups in proportions not exceeding 15% of the sum p+q+r;

i) quaternary ammonium groups, as in the products X2 8108® and X2 8109® and the product Abil K3270® from the company Goldschmidt;

j) amphoteric or betaine groups, as in the product sold by the company Goldschmidt under the name Abil B 9950®;

k) bisulfite groups, as in the products sold by the company Goldschmidt under the names Abil S 201® and Abil S 255®;

l) polyethyleneoxy and/or polypropyleneoxy groups optionally comprising C₆-C₂₄ alkyl groups, such as the products called dimethicone copolyol, sold by the company Dow Corning under the name DC 1248, or the oils Silwet L 722, L 7500, L 77 and L 711 from the company Union Carbide, and the (C₁₂)alkyl methicone copolyol sold by the company Dow Corning under the name Q2 5200.

According to the invention, use may also be made of silicones comprising a polysiloxane portion and a portion consisting of a non-silicone organic chain, one of the two portions constituting the main chain of the polymer, the other being grafted onto said main chain. These polymers are, for example, described in patent applications EP-A-412 704, EP-A-412 707, EP-A-640 105, WO 95/00578, EP-A-582 152 and WO 93/23009 and U.S. Pat. No. 4,693,935, U.S. Pat. No. 4,728,571 and U.S. Pat. No. 4,972,037. These polymers are preferably anionic or non-ionic.

Such polymers are, for example, copolymers that may be obtained by radical polymerization from the monomer mixture formed from:
a) 50% to 90% by weight of tert-butyl acrylate;
b) 0 to 40% by weight of acrylic acid;
c) 5% to 40% by weight of silicone macromer of formula (XVII):

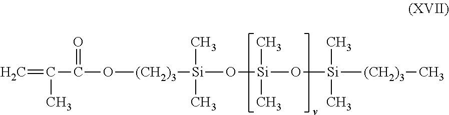

with v being a number ranging from 5 to 700; the percentages by weight being calculated relative to the total weight of the monomers.

Other examples of grafted silicone polymers are, in particular, polydimethylsiloxanes (PDMSs) onto which are grafted, by means of a connecting link of thiopropylene type, mixed polymer units of the poly((meth)acrylic acid) type and of the poly(alkyl(meth)acrylate) type; and polydimethylsiloxanes (PDMSs) onto which are grafted, by means of a connecting link of thiopropylene type, polymer units of the poly(isobutyl(meth)acrylate) type.

According to the invention, all the silicones can also be used in the form of emulsions, nanoemulsions or microemulsions.

The polyorganosiloxanes which are particularly preferred in accordance with the invention are:
non-volatile silicones selected from the family of polyalkylsiloxanes with trimethylsilyl end groups, such as oils having a viscosity of between 0.2 and 2.5 m²/s at 25° C., for instance the oils of the DC200 series from Dow Corning, in particular the one with a viscosity of 60 000 cSt, or of the Silbione 70047 and 47 series, and more particularly the oil 70 047 V 500 000 sold by the company Rhodia Chimie, and polyalkylsiloxanes with dimethylsilanol end groups, such as dimethiconols or polyalkylarylsiloxanes, for instance the oil Silbione 70641 V 200 sold by the company Rhodia Chimie;
polysiloxanes with amino groups, such as amodimethicones and trimethylsilyl amodimethicones.

The viscosities of the silicones may especially be determined by the standard ASTM D445-97 (viscometry).

When the conditioning agent of the composition according to the invention is a hydrocarbon, said hydrocarbon is a linear or branched C₈-C₃₀ hydrocarbon.

Among the hydrocarbons which are liquid at ambient temperature and which correspond to this definition, mention may in particular be made of isododecane, isohexadecane and its isomers (such as 2,2,4,4,6,6-heptamethylnonane), isoeicosane, isotetracosane, the isomers of said compounds, n-nonadecane, n-dodecane, n-undecane, n-tridecane, n-pentadecane, and mixtures of these hydrocarbons.

Use is preferably made according to the invention of isododecane or an isomer thereof.

When the conditioning agent is a fatty alcohol, said fatty alcohol is of the linear or branched, saturated or unsaturated C₈-C₃₀ type. Among the latter, mention may, for example, be made of 2-butyloctanol, lauryl alcohol, 2-octyldodecanol, oleyl alcohol, isocetyl alcohol, isostearyl alcohol and behenyl alcohol, and mixtures thereof.

When the conditioning agent is a fatty ester, said fatty ester may be either an ester of a C₈-C₃₀ fatty acid and of a C₁-C₃₀ alcohol, and in particular an ester of a C₈-C₃₀ fatty acid and of a C₈-C₃₀ fatty alcohol, or an ester of a C₁-C₇ acid or diacid and of a C₈-C₃₀ fatty alcohol.

Among these esters, mention may, for example, be made of ethyl, isopropyl, 2-ethylhexyl and 2-octyldecyl palmitate, isopropyl, butyl, cetyl and 2-octyldecyl myristate, butyl and hexyl stearate, hexyl and 2-hexyldecyl laurate, isononyl isononanoate, dioctyl malate, myristyl myristate and cetyl palmitate, and mixtures thereof.

The ceramides or ceramide analogues, such as glycoceramides, that can be used as conditioning agent in the compositions according to the invention are known in themselves and are natural or synthetic molecules that can correspond to general formula (XVIII) below:

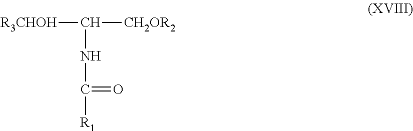

in which:
R₁ denotes a linear or branched, saturated or unsaturated alkyl group, derived from C₁₄-C₃₀ fatty acids, it being possible for this group to be substituted with a hydroxyl group in the alpha-position, or a hydroxyl group in the omega-position esterified with a saturated or unsaturated C₁₆-C₃₀ fatty acid;
R₂ denotes a hydrogen atom or a (glycosyl)ₙ, (galactosyl)ₘ or sulphogalactosyl group, in which n is an integer ranging from 1 to 4 and m is an integer ranging from 1 to 8;
R₃ denotes a C₁₅-C₂₆ hydrocarbon-based group, saturated or unsaturated in the alpha-position, it being possible for this group to be substituted with one or more C₁-C₁₄ alkyl groups;

it being understood that, in the case of natural ceramides or glycoceramides, R₃ can also denote a C₁₅-C₂₆ alpha-hydroxyalkyl group, the hydroxyl group being optionally esterified with a C₁₆-C₃₀ alpha-hydroxy acid.

The ceramides which are preferred in the context of the present invention are those described by Downing in Arch. Dermatol., Vol. 123, 1381-1384, 1987, or those described in French patent FR 2 673 179.

The ceramide(s) more particularly preferred according to the invention is (are) the compounds for which R₁ denotes a saturated or unsaturated alkyl derived from C₁₆-C₂₂ fatty acids; R₂ denotes a hydrogen atom; and R₃ denotes a linear, saturated C₁₅ group.

Such compounds are, for example:
N-linoleoyldihydrosphingosine,
N-oleoyldihydrosphingosine,
N-palmitoyldihydrosphingosine,
N-stearoyldihydrosphingosine,
N-behenoyldihydrosphingosine,

or mixtures of these compounds.

Even more preferably, ceramides for which R₁ denotes a saturated or unsaturated alkyl group derived from fatty acids; R₂ denotes a galactosyl or sulphogalactosyl group; and R₃ denotes a —CH═CH—(CH₂)₁₂—CH₃ group, are used.

By way of example, mention may be made of the product consisting of a mixture of these compounds, sold under the trade name Glycocer by the company Waitaki International Biosciences.

Among all these conditioning agents, use is preferably made of one or more conditioning agents selected from silicones such as organosiloxanes and cationic polymers.

The cosmetic composition according to the invention preferably contains from 0.01% to 20% by weight, and more preferably from 0.05% to 10% by weight of conditioning agent(s) relative to the total weight of the composition.

The composition according to the invention may also comprise at least one additional surfactant selected from non-ionic surfactants.

Examples of non-ionic surfactants that can be used in the composition used according to the invention are described, for example, in “Handbook of Surfactants” by M. R. PORTER, published by Blackie & Son (Glasgow and London), 1991, pp. 116-178. They are selected in particular from alcohols, alpha-diols, (C₁₋₂₀)alkylphenols or polyethoxylated, polypropoxylated and/or polyglycerolated fatty acids having a fatty chain containing, for example, from 8 to 18 carbon atoms, it being possible for the number of ethylene oxide and/or propylene oxide groups to range in particular from 2 to 50 and it being possible for the number of glycerol groups to range in particular from 2 to 30.

Mention may also be made of copolymers of ethylene oxide and of propylene oxide, optionally oxyethylenated fatty acid esters of sorbitan, fatty acid esters of sucrose, polyoxyalkylenated fatty acid esters, optionally oxyalkylenated alkylpolyglycosides, alkylglucoside esters, N-alkylglucamine and N-acylmethylglucamine derivatives, aldobionamides and amine oxides.

Unless otherwise mentioned, the term “fatty” compound (for example a fatty acid) denotes a compound comprising, in its main chain, at least one saturated or unsaturated alkyl chain containing at least 8 carbon atoms, preferably from 8 to 30 carbon atoms, and even better still from 10 to 22 carbon atoms.

When they are present, the amount of the non-ionic surfactant(s) preferably ranges from 0.01% to 20% by weight and better still from 0.2% to 10% by weight relative to the total weight of the composition.

Advantageously, the total content of anionic and amphoteric or zwitterionic surfactant(s), and optionally non-ionic surfactant(s), when it (they) is (are) present in the composition according to the invention, ranges from 4% to 50% by weight, and better still from 4% to 30% by weight, and more particularly from 10% to 20% by weight, relative to the total weight of the composition according to the invention.

The composition according to the invention is generally used in topical application.

In particular, the composition according to the invention may be in any galenical form normally used for topical application. The composition according to the invention may be a rinse-out or leave-in composition. In particular, it may be a shampoo, a cream, a gel or an emulsion. Preferably, the composition according to the invention is a shampoo.

The composition according to the invention comprises a cosmetically acceptable medium. The medium consists of water and optionally of one or more cosmetically acceptable organic solvents.

The organic solvent(s) may be selected from C₁-C₄ lower alcohols, such as ethanol, isopropanol, tert-butanol or n-butanol; polyols, such as glycerol, propylene glycol, hexylene glycol (or 2-methyl-2,4-pentanediol) and polyethylene glycols; polyol ethers, for instance dipropylene glycol monomethyl ether; and mixtures thereof.

When the medium comprises at least one cosmetically acceptable organic solvent, said solvent may be present in a proportion of from 0.1% to 30% by weight, preferably from 0.2% to 15% by weight, of the total weight of the composition.

The cosmetic composition according to the invention may have a pH of between 3 and 10 and preferentially between 5 and 7. This pH may be adjusted by means of acidifying and basifying agents conventionally used in cosmetics.

The cosmetic composition used according to the invention may also contain one or more adjuvants that are customary in the cosmetics field, such as agents for preventing hair loss, oxidizing agents, vitamins and provitamins, including panthenol, plant, animal, mineral or synthetic oils, waxes, sunscreens, coloured or uncoloured, mineral or organic pigments, dyes, pearlescent agents and opacifiers, sequestering agents, plasticizers, solubilizing agents, antioxidants, hydroxy acids, fragrances, anti-dandruff agents other than the compounds (i) as defined above, preservatives, and mixtures thereof.

The amounts of these various adjuvants are those conventionally used in the fields under consideration.

Of course, those skilled in the art will take care to select the optional compound(s) to be added to the composition according to the invention in such a way that the advantageous properties intrinsically associated with the composition in accordance with the invention are not, or not substantially, impaired by the addition(s) envisaged.

Another subject of the invention consists of a cosmetic treatment method for the scalp and for keratin fibres, in particular human keratin fibres such as the hair, comprising the application of a composition according to invention to the scalp and said keratin fibres. After an optional pause time preferably ranging from 1 to 15 minutes, the composition is then preferably rinsed out with water. Said method is preferably intended for washing said keratin fibres and/or for anti-dandruff treatment intended for eliminating and/or reducing dandruff, and in particular dandruff caused by yeasts of the Malassezia genus.

A subject of the invention is also the use of a composition according to the invention for washing keratin fibres, in particular human keratin fibres such as the hair.

A subject of the invention is also the use of a composition according to the invention for anti-dandruff treatment intended for eliminating and/or reducing dandruff, and in particular dandruff caused by yeasts of the Malassezia genus.

The examples which follow are intended to illustrate the invention without, however, being limiting in nature.

### EXAMPLES

### Example 1

### Shampoo

The following composition was prepared. The amounts are indicated as percent by weight of active material (A.M.) relative to the total weight of the composition.

| | % by weight |
|---|---|
| Composition | of A.M. |
| Fragrance | 0.5 |
| Coconut acid monoisopropanolamide | 0.25 |
| (Empilan CIS sold by the company Huntsman) | |
| Cocoylamidopropylbetaine at 38% by weight | 2.4 |
| in an aqueous solution (Tego betain F50 sold | |
| by the company Evonik Goldschmidt) | |
| Sodium lauryl ether sulphate containing | 15.5 |
| 2.2 mol of ethylene oxide, at 26% by weight | |
| in an aqueous solution (Texapon AOS 225 UP | |
| sold by the company Cognis) | |
| Ellagic acid | 1 |
| Sodium benzoate | 0.5 |
| Salicylic acid | 0.2 |
| Polydimethylsiloxane 500 000 cSt (molecular | 2 |
| weight 250 000) (Silbione 70047 V500000 | |
| sold by the company Bluestar) | |
| 1-(Hexadecyloxy)-2-octadecanol/cetyl | 2.5 |
| alcohol mixture (Mexanyl GY sold by the | |
| company Chimex) | |
| Carboxyvinyl polymer (Carbopol 980 sold by | 0.3 |
| the company Lubrizol) | |
| Propylene glycol | 0.1 |
| Glycerol | 2 |
| Sodium chloride | 1.1 |
| pH agents | qs pH 5.3 |
| Fragrance | Qs |
| Water | qs 100 |

The shampoo obtained has the appearance of a viscous opaque beige liquid, and is characterized by a fine dispersion of the ellagic acid and is stable over time.

Applied to the hair and the scalp and then rinsed out, the shampoo exhibits good usage qualities (easy to apply, pleasant and soft foam, easy to remove), promoting distribution of the ellagic acid on the scalp while at the same time exhibiting good cosmetic effectiveness on the hair (softness, smoothing) associated with good detergence.

Anti-dandruff effectiveness is good, in particular with repeated application.

### Example 2

### Shampoo

The following composition was prepared. The amounts are indicated as percent by weight of active material (A.M.) relative to the total weight of the composition.

| | % by weight |
|---|---|
| Composition | of A.M. |
| Polydimethylsiloxane (Belsil DM 60 000 sold | 0.7 |
| by the company Wacker) | |
| Coconut acid monoisopropanolamide | 0.5 |
| (Empilan CIS sold by the company Huntsman) | |
| Cocoylbetaine at 30% by weight in an | 1.8 |
| aqueous solution (Dehyton AB 30 sold by the | |
| company Cognis) | |
| Carboxyvinyl polymer (Carbopol 980 sold by | 0.2 |
| the company Lubrizol) | |
| Hydroxypropyl guar trimethylammonium | 0.03 |
| chloride (Jaguar C13-S sold by the company | |
| Rhodia) | |
| Ellagic acid | 1 |
| Ethylene glycol distearate | 1.6 |
| Salicylic acid | 0.2 |
| Sodium benzoate | 0.5 |
| Sodium lauryl ether sulphate containing | 12.2 |
| 2.2 mol of ethylene oxide, at 26% by weight | |
| in an aqueous solution (Texapon AOS 225 UP | |
| sold by the company Cognis) | |
| Hexylene glycol (2-methyl-2,4-pentanediol) | 0.5 |
| Sodium chloride | 1.5 |
| pH agents | qs pH 5.3 |
| Fragrance | Qs |
| Water | qs 100 |

The shampoo obtained has the appearance of a viscous opaque beige liquid, and is characterized by a fine dispersion of the ellagic acid and is stable over time.

Applied to the hair and the scalp and then rinsed out, this shampoo exhibits good usage qualities (easy to apply, pleasant and soft foam, easy to remove), promoting distribution of the ellagic acid on the scalp while at the same time exhibiting good cosmetic effectiveness on the hair (softness, smoothing) associated with good detergence.

Anti-dandruff effectiveness is good, in particular with repeated application.

### Example 3

### Shampoo

The following composition was prepared. The amounts are indicated as percent by weight of active material relative to the total weight of the composition.

| | % by weight |
|---|---|
| Composition | of A.M. |
| Sodium lauryl sulphate | 6.75 |
| Sodium lauryl ether sulphate containing | 8.25 |
| 2.2 mol of ethylene oxide | |
| Glycerol | 2 |
| Sodium chloride | 1.2 |
| Ellagic acid | 1 |
| Ethylene glycol distearate | 1 |
| Salicylic acid | 0.2 |
| Coconut acid monoisopropanolamide | 2 |
| (Comperlan 100 sold by the company Cognis) | |
| Polydimethylsiloxane (Mirasil DM 300 000 | 0.4 |
| sold by the company Rhodia) | |
| Sodium benzoate | 0.5 |
| Hydroxypropyl guar trimethylammonium | 0.05 |
| chloride (Jaguar C13-S sold by the company | |
| Rhodia) | |
| Carboxyvinyl polymer (Carbopol 980 sold by | 0.3 |
| the company Lubrizol) | |
| Fragrance, dye | qs |
| Water | qs 100 |

The shampoo obtained exhibits a fine dispersion of the ellagic acid and is stable over time.

Applied to the hair and the scalp, this shampoo exhibits good usage qualities (easy to use, pleasant and soft foam, easy to remove), promoting distribution of the ellagic acid on the hair and the scalp while at the same time exhibiting good cosmetic effectiveness (softness, smoothing) associated with the good detergence. Anti-dandruff effectiveness is good, in particular with repeated application.

1. Cosmetic composition comprising, in a cosmetically acceptable medium:
(i) from 0.2% to 10% by weight of one or more compounds selected from ellagic acid, ethers thereof, ellagic acid salts and ellagic acid ether salts, and mixtures thereof, and
(ii) a system of surfactants selected from the following mixtures comprising:
(iii) one or more anionic surfactants selected from ethoxylated alkyl sulphates, the corresponding acids, and mixtures thereof, and (iv) one or more anionic surfactants different from the abovementioned anionic surfactant(s) (iii), and
(v) one or more anionic surfactants and (vi) one or more amphoteric or zwitterionic surfactants, in a weight ratio of the amount of anionic surfactant(s) to the amount of amphoteric or zwitterionic surfactant(s) of greater than 3.

2. Composition according to claim 1, characterized in that the salt(s) of the compound (i) is (are) selected from the alkali metal or alkaline-earth metal salts, in particular the sodium, potassium, calcium or magnesium salts.

3. Composition according to claim 1, characterized in that the ellagic acid ether(s) is (are) chosen from mono-, di-, tri- or polyethers obtained by etherification of one or more hydroxyl groups of ellagic acid to give one or more OR groups, R being selected from C₂-C₂₀ alkyl groups, polyoxyalkylene groups, and in particular polyoxyethylene and/or polyoxypropylene groups, and groups derived from one or more monosaccharides or polysaccharides.

4. Composition according to claim 1, characterized in that the compound (i) is ellagic acid or a salt thereof.

5. Composition according to claim 1, characterized in that it comprises from 0.2% to 5% by weight, and even better still from 0.2% to 2% by weight of compound(s) (i), relative to the total weight of the composition.

6. Composition according to claim 1, characterized in that the anionic surfactant(s) (iii) is (are) selected from ethoxylated (C₆-C₂₄)alkyl sulphates comprising from 1 to 50 mol of ethylene oxide, preferably from 1 to 10 mol, in particular from 1 to 5 mol, and even better still from 2 to 3 mol.

7. Composition according to claim 1, characterized in that it comprises from 0.1% to 50% by weight, in particular from 2% to 20% by weight, even better still from 4% to 15% by weight, and even better still from 5% to 15% by weight of anionic surfactant(s) (iii), relative to the total weight of the composition.

8. Composition according to claim 1, characterized in that the anionic surfactant(s) (iv) different from the anionic surfactants (iii) is (are) selected from nonethoxylated alkyl sulphates, alkylamido ether sulphates, alkylaryl polyether sulphates, monoglyceride sulphates, alkyl sulphonates, alkylamide sulphonates, alkylarylsulphonates, alpha-olefin sulphonates, paraffin sulphonates, alkylsulphosuccinates, alkyl ether sulphosuccinates, alkylamide sulphosuccinates, alkyl sulphoacetates, acylsarcosinates, acylglutamates, alkyl sulphosuccinamates, acyl isethionates and N-acyltaurates; salts of alkyl monoesters of polyglycoside-polycarboxylic acids, acyllactylates, salts of D-galactosiduronic acids, salts of alkyl ether carboxylic acids, salts of alkyl aryl ether carboxylic acids, salts of alkylamido ether carboxylic acids; and the corresponding non-salified forms of all these compounds; the alkyl and acyl groups of all these compounds containing from 6 to 24 carbon atoms and the aryl group denoting a phenyl group.

9. Composition according to claim 1, characterized in that it comprises from 0.1% to 30% by weight, in particular from 1% to 20% by weight, and even better still from 2% to 15% by weight of anionic surfactant(s) (iv).

10. Composition according to claim 1, characterized in that the weight ratio of the amount of anionic surfactant(s) (iii) to the amount of anionic surfactant(s) (iv) ranges from 0.25 to 20, preferably from 0.5 to 10, better still from 1 to 5, and even better still from 1 to 2.5.

11. Composition according to claim 1, characterized in that the anionic surfactant(s) (v) is (are) selected from alkyl sulphates, alkyl ether sulphates, alkylamido ether sulphates, alkylaryl polyether sulphates, monoglyceride sulphates, alkyl sulphonates, alkylamide sulphonates, alkylarylsulphonates, alpha-olefin sulphonates, paraffin sulphonates, alkylsulphosuccinates, alkyl ether sulphosuccinates, alkylamide sulphosuccinates, alkyl sulphoacetates, acylsarcosinates, acylglutamates, alkyl sulphosuccinamates, acyl isethionates and N-acyltaurates; salts of alkyl monoesters of polyglycoside-polycarboxylic acids, acyllactylates, salts of D-galactosiduronic acids, salts of alkyl ether carboxylic acids, salts of alkyl aryl ether carboxylic acids, salts of alkylamido ether carboxylic acids; and the corresponding non-salified forms of all these compounds; the alkyl and acyl groups of all these compounds containing from 6 to 24 carbon atoms and the aryl group denoting a phenyl group.

12. Composition according to claim 1, characterized in that it comprises from 0.1% to 50% by weight, in particular from 4% to 30% by weight, and even better still from 8% to 20% by weight of anionic surfactant(s) (v).

13. Composition according to claim 1, characterized in that the amphoteric or zwitterionic surfactant(s) (vi) is (are) selected from (C₈₋₂₀)alkylbetaines, sulphobetaines, (C₈₋₂₀ alkyl)amido(C₂₋₈ alkyl)betaines, and (C₈₋₂₀ alkyl)amido(C₂₋₈ alkyl)sulphobetaines and preferably from (C₈₋₂₀ alkyl)betaines, and (C₈₋₂₀ alkyl)amido(C₂₋₈ alkyl)betaines.

14. Composition according to claim 1, characterized in that the amphoteric or zwitterionic surfactant(s) (vi) is (are) selected from the compounds of respective structures (A2) and (A3) below:
Rₐ—CONHCH₂CH₂—N⁺(R_{b})(R_{c})(CH₂COO⁻) (A2)
in which:
Rₐ represents a C₁₀-C₃₀ alkyl or alkenyl group derived from an acid Rₐ—COOH preferably present in hydrolysed coconut oil, a heptyl group, a nonyl group or undecyl group,
R_{b} represents a beta-hydroxyethyl group, and
R_{c} represents a carboxymethyl group;
and
R_{a′}—CONHCH₂CH₂—N(B)(B′) (A3)
in which:
B represents —CH₂CH₂OX′,
B′ represents —(CH₂)_{z}—Y′, with z=1 or 2,
X′ represents the —CH₂—COOH, CH₂—COOZ′, —CH₂CH₂—COOH or —CH₂CH₂—COOZ′ group, or a hydrogen atom,
Y′ represents —COOH, —COOZ′, or the —CH₂—CHOH—SO₃H or —CH₂—CHOH—SO₃Z′ group,
Z′ represents an ion derived from an alkali or alkaline-earth metal, such as sodium, potassium or magnesium; an ammonium ion; or an ion derived from an organic amine and in particular from an aminoalcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane.
R_{a′} represents a C₁₀-C₃₀ alkyl or alkenyl group of an acid R_{a′}COOH preferably present in hydrolysed linseed oil or coconut oil, an alkyl group, which is in particular C₁₇, and its iso form, or an unsaturated C₁₇ group.

15. Composition according to claim 1, characterized in that it comprises from 0.01% to 20% by weight, in particular from 0.5% to 10% by weight, and even better still from 1% to 5% by weight of amphoteric or zwitterionic surfactant(s) (vi), relative to the total weight of the composition.

16. Composition according to claim 1, characterized in that it comprises one or more thickeners and/or one or more conditioning agents preferably selected from silicones and cationic polymers, and/or one or more additional surfactants selected from non-ionic surfactants.

17. Cosmetic treatment method, characterized in that it comprises the application of a cosmetic composition as defined in claim 1 to the scalp and to keratin fibres, in particular human keratin fibres such as the hair.

18. Use of a cosmetic composition as defined in any claim 1 for washing keratin fibres, in particular human keratin fibres such as the hair.
